(19) United States
(12) Patent Application Publication
(10) Pub. No.: US 2013/0068233 A1
(43) Pub. Date: Mar. 21, 2013

(54) ENDOTRACHEAL TUBE STABILIZER APPARATUS AND METHOD

(71) Applicants: David Mark De Lulio, Lafayette, LA (US); Donald Lee Morris, Littleton, CO (US); James Ringgold Strong, Clayton, MO (US); James Ringgold Strong, JR., St. Louis, MO (US)

(72) Inventors: David Mark De Lulio, Lafayette, LA (US); Donald Lee Morris, Littleton, CO (US); James Ringgold Strong, Clayton, MO (US); James Ringgold Strong, JR., St. Louis, MO (US)

(73) Assignee: VCB IP Holdings, LLC, Clayton, MO (US)

(21) Appl. No.: 13/614,348

(22) Filed: Sep. 13, 2012

Related U.S. Application Data
(63) Continuation-in-part of application No. 13/231,423, filed on Sep. 13, 2011.

(51) Int. Cl. A61M 16/04

(52) U.S. Cl. CPC A61M 16/0497
USPC 128/207.17

(57) ABSTRACT

A neonatal endotracheal tube stabilizer has a tube cradle, a tube fixation element attached to said tube cradle and a stabilization bar having a plurality of engagement elements. The stabilization bar extends on either side of the tube cradle to engage two cheek pads. The cheek pads each have a releasable engagement interface dimensioned to engage the engagement elements of the stabilization rod in at least one engaged position. When so engaged, the cradle firmly holds the endotracheal tube in a position selected by a health care provider.

### RELATED APPLICATIONS

This non-provisional application is a continuation-in part of U.S. patent application Ser. No. 13/231,423, titled ENDOTRACHEAL TUBE STABILIZER APPARATUS AND METHOD, filed Sep. 13, 2011. This application incorporates by reference all of the disclosures therein.

### FIELD OF THE INVENTION

The invention is in the field of medical devices, particularly position and stabilization equipment for endotracheal tubes.

### BACKGROUND OF THE INVENTION

Few things in medical care are as sensitive and demanding of precision for health care personnel as neonatal intubation. Proper placement of the newborn's “breathing tube,” ease, speed and accuracy of positioning, removal and replacement are of paramount importance. Accordingly, certain prior art devices have been developed to advance the art past simple taping of the tube to the newborn. However, there remains a need in the art for devices which improve the ease, speed and accuracy of positioning, placement, removal and replacement of endotracheal tubes for neonates. There is a further need for devices that protect the skin of the child and for equipment that facilitates potential emergent response to changes in the child's status.

### SUMMARY OF THE INVENTION

The neonatal endotracheal tube stabilizer of the present invention has a tube cradle, a tube fixation element attached to said tube cradle and a stabilization bar having a plurality of engagement elements. The stabilization bar extends on either side of the tube cradle to engage two cheek pads. The cheek pads each have a releasable engagement interface dimensioned to engage the engagement elements of the stabilization rod in at least one engaged position. When so engaged, the cradle firmly holds the endotracheal tube in a position selected by a health care provider.

The engagement designs of both the tube cradle and between the stabilization rod and cheek pads provide for advantageous ease of use, flexibility of placement, and rapidity of release or readjustment.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 is the exploded diagram of the endotracheal tube stabilizer of the present invention.

FIG. 2 is close up perspective view of a cheek pad.

FIG. 3 is a perspective view of the support bar and tube cradle of the present invention.

FIG. 4 is a top view of the support bar and tube cradle.

FIG. 5 is an isometric view of a part of another implementation.

FIG. 6 is an isometric view of a part of another implementation.

FIG. 7 is an isometric view of a part of another implementation.

FIG. 8 is an isometric view of a part of another implementation

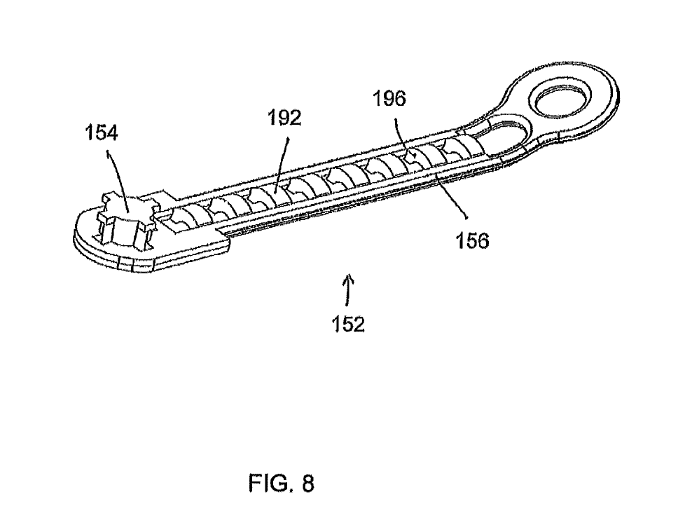

FIG. 9 is a perspective view of another implementation of the support bar and tube cradle of the present invention.

FIG. 10 is a perspective view of another implementation of a cheek pad of the present invention.

FIGS. 11A and 11B illustrate perspective views of additional implementations having support bars of differing lengths.

### DETAILED DESCRIPTION OF THE PREFERRED EMBODIMENTS

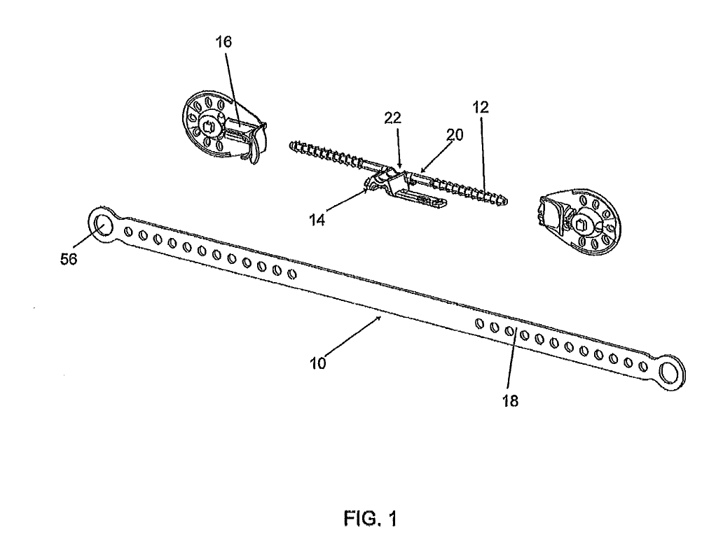

Referring now to the drawings in which like reference numbers indicate like elements, FIG. 1 is an exploded view of the neonatal endotracheal tube stabilizer of the present invention 10. The components of the invention include the support bar 12, the endotracheal tube (“ET Tube”) cradle 14, two cheek pads 16 and a neck strap 18.

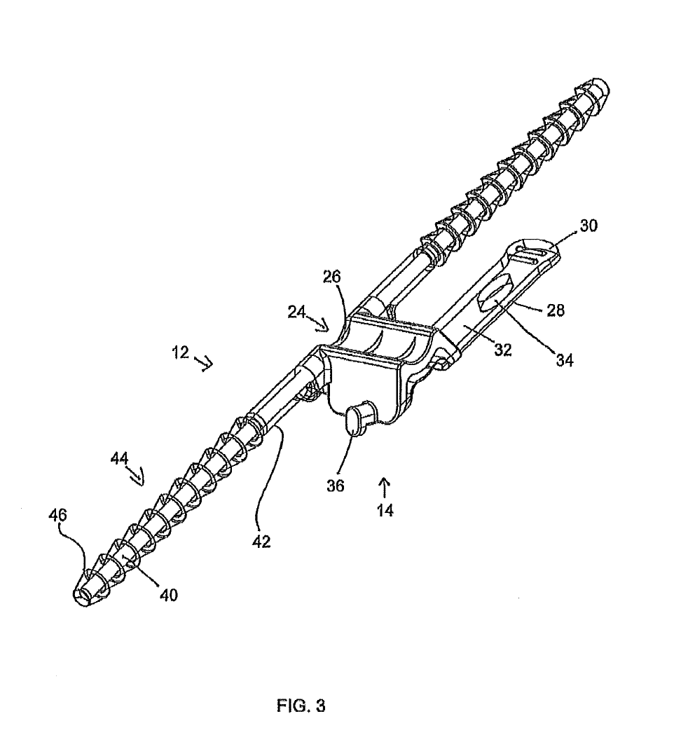

FIG. 3 depicts the support bar (or stabilization bar) 12 and tube cradle 14 as assembled. The assembly of the ET Tube cradle 14 with the support bar 12 is by the engagement of ET Tube cradle slot 20 with central detent 22 in the support bar 12. Detent 22, as depicted, is substantially U-shaped and oriented downwards. This achieves simultaneously the effect of creating an appropriately dimensioned space for passage of the endotracheal tube, as well as an engagement with the ET Tube cradle 14 that is stable in multiple dimensions. Because the detent 22 is offset from the longitudinal axis of the support bar 12, which is oriented to be substantially transverse to the air passage or mouth of the patient, the detent 22 further promotes proper positioning of the ET Tube at the desired central position aligned with the patient's airway.

The ET Tube cradle 14 includes a concavity 24 on one aspect, which, in the depicted embodiment, opens upwards or towards the patient's nose. This defines a seat on which the endotracheal tube may rest and be stabilized. The seat/concavity 24 may be equipped with ribs 26 to arrest motion of the ET Tube inwards or outwards by friction when the ET Tube is fixed in position.

The ET Tube is fixed in its position in the ET Tube cradle 14, in the depicted embodiment, by means of a tube fixation element, such as a strap 28. Strap 28 may be fabricated of elastomeric material having some elasticity. The strap 28 is comprised of a thumb and finger tab 30, a stretchable ET Tube contact portion 32 and a through hole 34. In operation, after the tube stabilization device has been properly positioned and the ET Tube inserted into the airway to its proper depth, the strap 28 is stretched slightly outwards, wrapped over the top of the ET Tube on the ET Tube cradle 14 and then hole 34 is engaged with tab 36 to secure it in a locking position when released. The elasticity of the strap 28 serves to hold the hole 34 secure against the tab 36 and create pressure that holds the ET Tube securely in position against both lateral and axial movement relative to the ET Tube cradle 14.

In the depicted embodiment the support bar 12 may be fabricated of a malleable, semi-rigid material, for example brass or stainless steel, with an elastomeric over mold. Accordingly, metal rod 40 is covered with the elastomeric material 42.

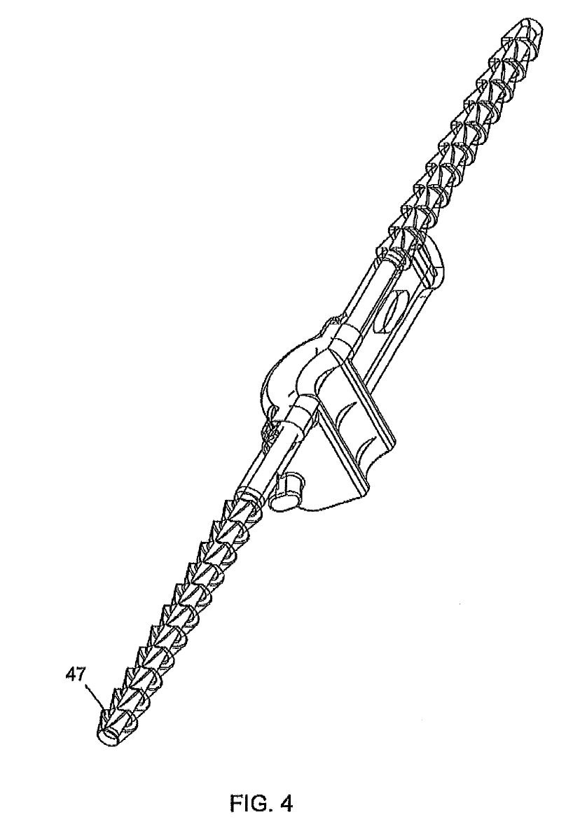

The support bar 12 is made of malleable material so that the left and right wings 44 may be bent around the mouth and cheeks of the patient. An originally straight and malleable configuration allows for ease of manufacturing and customizable shaping for each patient. The elastomeric material over the metal is configured to have at least one and, as depicted, a plurality of engagement elements 46. In the depicted embodiment these engagement elements 46 are teeth, which may be frustoconical. The depicted teeth are oriented with their narrow end outwards, to promote ease of assembly with the cheek pads 16. Of note and best seen in FIG. 4, the teeth are not fully circumferential around the stabilizing bar 40. This axially truncated shape 47 further promotes the proper orientation and positioning of the ET Tube cradle 14 relative to the patient's airway, due to the manner in which the flat side of the teeth engages with the corresponding structure in the cheek pads 16 as more fully described below.

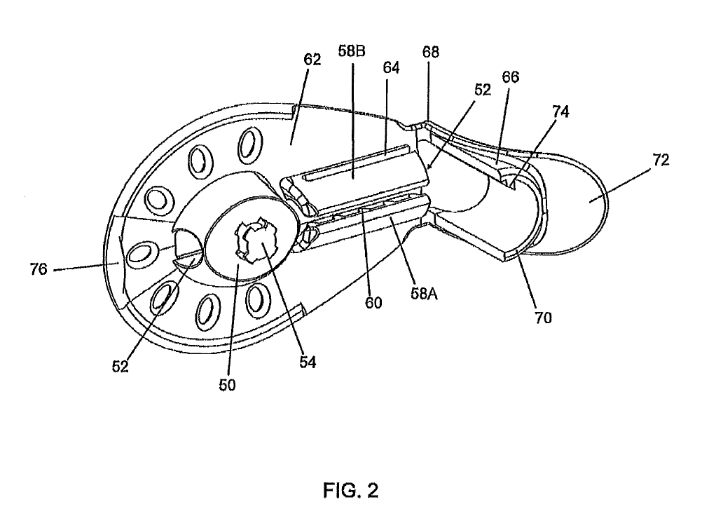

FIG. 2 depicts the cheek pad 16. An opposing side of the cheek pad away from view in FIG. 2 is substantially flat and configured to receive an interfacing material for contact with the patient's skin, such as an adhesive mole skin backing. The outwardly facing side of the cheek pad 16 includes the following components. A support bar receiving element 50 includes a passage 52 for receiving the support bar 12. In the depicted embodiment a neck strap post 54 is located on top of the receiving element 50. The neck strap post 54 is a positive element designed to engage the hole 56 at the end of the neck strap 18 (or any of the intermediate holes in the neck strap) for further tractive stabilization of the overall device. As the name implies, the neck strap 18 stretches from engagement of a left end hole 56 with a left cheek pad neck strap post 54 around the back of the patient's head to an opposing right side neck strap hole(s) 56 with the corresponding neck strap post 54 on the right cheek pad 16.

The bar retainer 58 defines the passage 52 into which the support bar 12 is inserted during assembly on the patient. In the depicted embodiment the receiver portions 58A and 58B includes teeth 60 configured and dimensioned to interlock with the spaces between the teeth or engagement elements 46 on the support bar 12. The teeth 60 on the cheek pad extend circumferentially around the inner surface of the retainer 58. However, in the depicted embodiment there are no teeth 60 on the surface of the outer planar surface 62 of the cheek pad that is located within the channel 52 for retaining the support bar. Hence the untoothed flat surface 62 matches with the flat lateral truncations 47 of the teeth or locking elements 46 in order to positively align the support bar 12 rotationally, which again promotes positioning of the ET Tube cradle 14 with the desired position aligned with the patient's airway. In the depicted embodiment the receiver 58 is bifurcated into receiver wings 58A and 58B. Since the cheek pad 16 as depicted is molded of an elastomeric material, this gives the receiver 58 flexibility so that the teeth 60 may ride up and over the engaging elements 46 upon insertion of the support bar 12. Once the proper degree of insertion of the support bar 12 through the receiver 58 has been reached for an appropriate custom fit with a particular patient, the elasticity of the receiver wings 58A, 58B will bias them inwards towards a retaining, engaged position of teeth 60 with engaging elements 46. Receiver wings 58A and 58B, may have a thin portion molded into them, as represented by slot 64, to engage boss 74 on bar lock 66 and close channel 52 and secure bar 12.

Once the support bar 12 has been inserted into the receiver 58 of the cheek pad 16 to an appropriate position, that position may be locked into a holding engagement without further flexion of the receiver 58 by the use of the bar lock 66. Bar lock 66 is attached to the rest of the cheek pad 16 by a moveable element which, as depicted, may be a hinge 68. The bar lock 66 includes an encapsulating semicircle 70 dimensioned to fit over the outer surface of the receiver 58. A finger tab 72 facilitates its actuation by an operator. A boss 74 is dimensioned to engage slot 64 for positive locking into position. A symmetric cheek pad 16 is similarly assembled on the other side of the patient. Extension 76 serves as a finger tab and an end stop against over insertion of support bar 12.

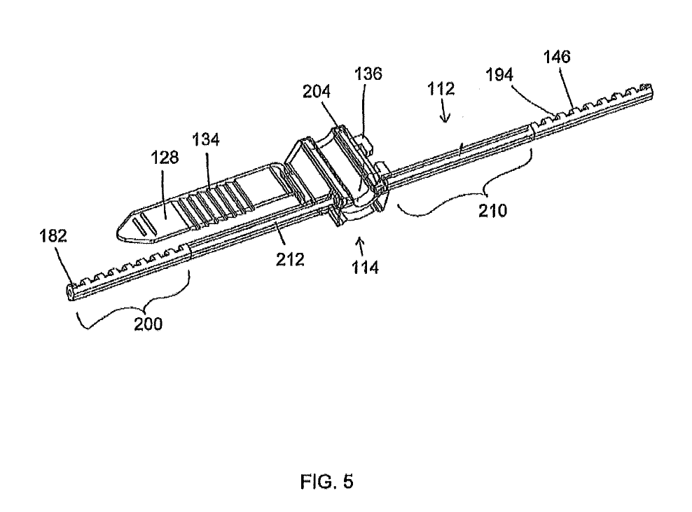

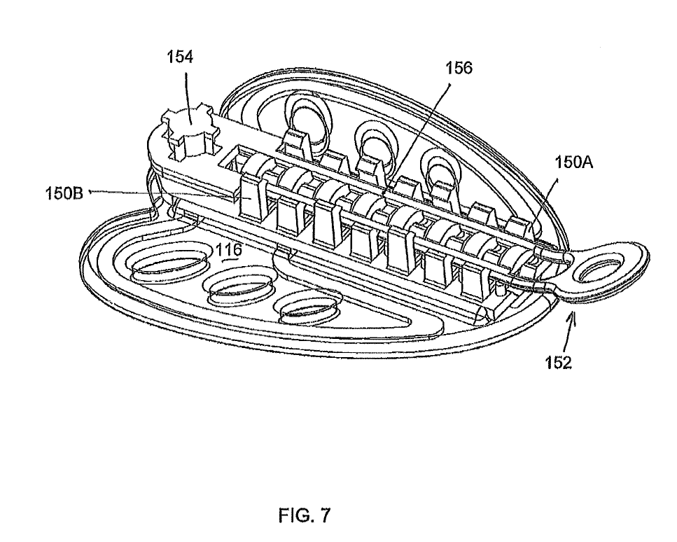

In an alternative embodiment, depicted in FIG. 5 the support bar 112 has alternative engagement elements 146. The cradle 114 and retaining strap 128 are substantially similar to the embodiment described above, with the exception that retaining means 134 comprise bosses for engagement in a receiving structure 136. The cheek pads 116 in the alternate embodiment include a plurality of retaining elements 150A, 150B each comprised of a displaceable material such as plastic that is biased to return to and remain in a retaining position such as that depicted in FIG. 7. The retaining elements 150A, 150B are dimensioned to engage the retaining elements 146 on the rod 112.

The cheek pad 116 of this embodiment has three positions. In the engaged or closed position, those retaining elements 150A, 150B having the shorter vertical dimension 150A will retain a longitudinal locking element 152 in position to hold the stabilizer bar 112 on the cheek pad. Another position is completely removed from engagement, which position is provided for emergency removal of the bar 112 and cradle 114 apparatus. In such a circumstance, the longitudinal members 156 of the locking element 152 may be displaced inwardly for disengagement with the hooking elements of the retaining elements 150A, 150B, thereby making the entire locking element 152 removable from the cheek pad and thereafter, the rod 112 also removable therefrom. In an intermediate position, taller retaining elements 150B are used to temporarily engage the longitudinal elements 156 of the retaining strap 152. In this position, the stabilizer bar 112 may be translated through the channel 184 defined by the series of retaining elements 150A, 150B in the cheek pad 116. This position is used for longitudinal adjustment the bar 112 and cheek pad 116 relative to one another, in order for the user to find an optimal position for the endotracheal stabilizer overall. Once the ideal position of the stabilizer bar 112 relative to the cheek pad 116 has been selected, the longitudinal elements 156 of the locking element 152 are pressed into the lower-most, retaining position to fix the user-selected position for ongoing placement and stabilization of an endotracheal tube. One end of locking element 152 extends beyond the check pad 116 as a tab to facilitate positioning or removal by being easier to grasp.

In the cheek pad assembly a post 178 is provided. In the depicted embodiment the post is somewhat conical and smooth. The outside dimension of post 178 matches the inner dimensions of post receiver 154 in retaining strap 152. The outer dimensions of post receiver 154 are used for mounting and seating a neck strap hole for adjustment of the neck strap. Any one of the adjustment holes in the neck strap may be placed over elements 54 or 154 for appropriate adjustment.

The smooth interface in the depicted embodiment between the outer surface of post 178 and the inner surface of post receiver 154 facilitates ease of vertical adjustment of the retaining strap 152 among its three different positions.

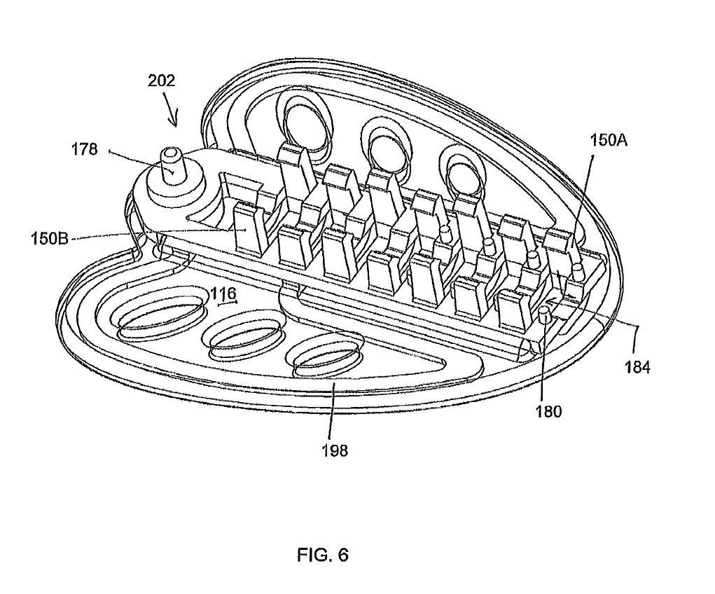

FIG. 6 further depicts retaining posts 180 that project upwardly into a channel defined for receiving the stabilizing bar 112. After assembly, the innermost of these retaining posts 180—that is, the retaining post 180 most distal from the patient's mouth—will engage tabs 182 in an end area of the stabilizer bar 112. By abutting the retaining posts 180, the tabs 182 arrest separation or axial movement of the stabilizer bar 112 outwards from the channel 184 defined for it in the cheek pad receiver assembly.

To facilitate sanitary packaging and ease of application of the device on the patient, the nurse receives the stabilizer bar and cheek pad in a pre-assembled position within a sealed package which may optionally be sterile. The cheek pad/stabilizer bar assembly is packaged in the intermediate position, ready for adjustment to the individual patient as described above, but not needing to be assembled together. The tube holder device is also removed from its sealed packaging with a cover over the adhesive surface on the inside of the cheek pads. The adjustment procedure allows for adjustments to be made and the stabilizer bar, retaining strap and cheek pad receiver assembly to be placed in their lockdown position before removal of the adhesive cover and application of the adhesive inner surface of the cheek pad to the patient.

Upon pressure downwardly on the retaining strap 152 in order to place the longitudinal elements 156 in the lockdown position, the outwardly facing bosses 146 of the stabilizer bar 112 will engage the rungs or transverse members 192 of the retaining strap 152. Hence, the stabilizer bar 112 is restrained from being withdrawn improperly from the cheek pad by the abutment of a proximal side 194 of each boss 146 with a distal side 196 of the transverse members 192 of the retaining strap 152. As a further assurance against improper withdrawal, the tabs 182 would be arrested from improper withdrawal by pegs 180 in any event. Before the longitudinal elements 156 of the retaining strap 152 are placed in their lockdown position, the tabs 182 do not interfere with longitudinal travel of the stabilizer bar 112 in the receiving channel 184.

The overall cheek pad assembly flexes and is bendable to facilitate a comfortable adjustment and placement on the patient in a manner that enables the device to be contoured to the patient's facial features. An appropriate amount of rigidity is maintained by a stiffer skeleton element 198 within the cheek pad.

The cradle or sled for receiving the endotrachael tube in the depicted embodiments would have a friction surface for arresting undesirable travel of the endotrachael tube in or out of the patient's mouth. This may be by means of the ribs 26 depicted in the first embodiment or, in the alternative, may include, without limitation a soft elastomeric material having a higher coefficient of friction to serve the same purpose.

The present invention also includes an advantageous method of manufacture. The depicted embodiments are comprised of a malleable metal stabilizing rod core over which the various operational elements and features of the invention are molded in polymers. A harder polymer such as, for example without limitation, polyurethane, may be used where greater rigidity may be advantageous, for example the outer sections of the stabilizing rod 112 such as at area 200 having the teeth 46/146 and in the stabilizer bar receiver assembly 202 on the cheek pads 116. The same is true for inner components 204 of the tube cradle. The same may be true for tube strap receiver 136. Other portions of the device of the present invention may be more advantageously made of a soft material such as a more flexible elastomeric, including for example portion 210 of the stabilizer bar and strap 128.

According to the method of manufacturing the present invention, the rigid but malleable rod 212 would be placed in a first mold or a first concave of a single mold having a first insert that would block or fill the space to be later filled by the softer elastomeric material. The tool would then define as its inner concavity the shape of the harder plastic elements 200 (or 202). These would be injected and molded. After cooling and extraction of the stabilizer rod core 212 with the hard plastic components now attached, the work piece is placed in a second mold, or a second portion of the same mold, having a different internal concavity. The work piece assembly 212/200 is put in position in order to receive the injection of the softer elastomeric material, in order to fabricate portions 210 and the other soft polymer portions of the device. Advantageously, the structure of the bosses 146, tabs 182 of hard plastic in the portions 200 of the stabilizing rod 112 may be used to hold, stabilize and/or center the work piece in the mold for injection of the softer elastomeric material. Hence by over molding the rod 212 is properly positioned in the softer elastomeric material.

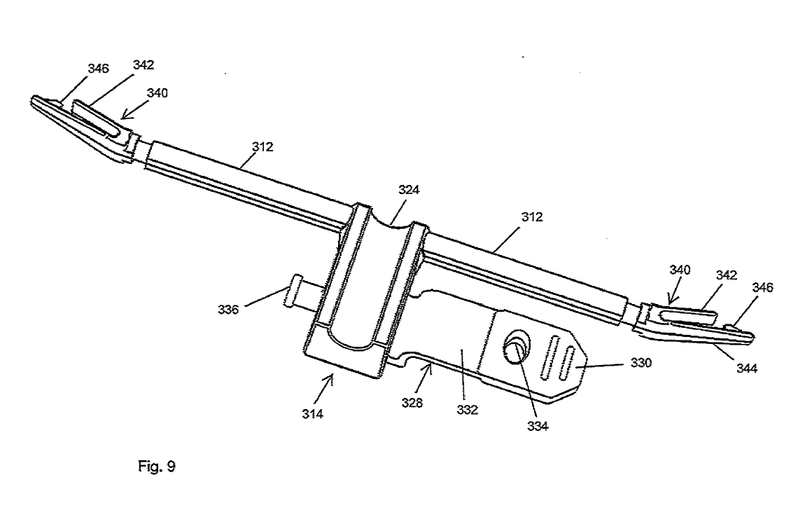

An alternative embodiment is shown in FIGS. 9-11. As can be seen in FIG. 9, this implementation includes a support bar 312 and tube cradle 314, as in the above embodiments. The tube cradle 314 similarly includes a concavity 324, which preferably opens upwards or towards the patient's nose. This defines a seat on which the endotracheal tube may rest and be stabilized. An ET Tube is preferably fixed in its position in the tube cradle 314 by strap 328. As above, strap 328 may be fabricated of elastomeric material having some elasticity. The strap 328 is comprised of a thumb and finger tab 330, a stretchable ET Tube contact portion 332 and a through hole 334. In operation, after the tube stabilization device has been properly positioned and the ET Tube has been inserted into the airway to its proper depth, the strap 328 is stretched slightly outwardly, wrapped over the top of the ET Tube in the tube cradle 14, and then hole 334 is engaged with tab 336 to secure it in a locking position when released. The elasticity of the strap 328 serves to hold the hole 334 securely against the tab 336 and create pressure that holds the ET Tube securely in position against both lateral and axial movement relative to the tube cradle 314.

As discussed above in connection with another implementation, the tube cradle 314 is preferably comprised of a material which has a high coefficient of friction, such as an elastomeric material. Such material helps to prevent unwanted movement of an endotracheal tube therein.

At the ends of support bar 312 is a clip 340 for selectively securing the support bar 312 to a cheek pad 316. Clip 340 is another example of an engagement element. Clip 340 includes a lower finger 342, which is separated by a gap from an upper finger 344. Upper finger 344 is preferably longer than lower finger 342, and upper finger 344 preferably includes a prong 346 extending downwardly therefrom into the gap between the upper and lower fingers 342, 344.

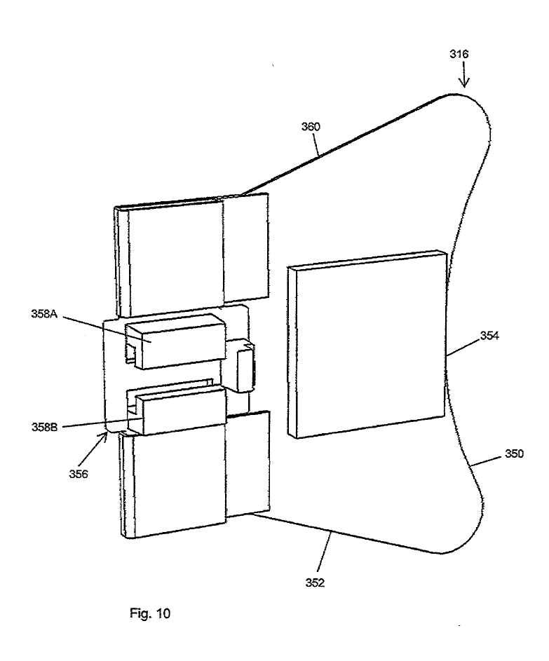

An exemplary cheek pad 316 is shown in FIG. 10. Cheek pad 316 includes an outside surface 350 and an inside surface 352. A strap (not shown in this embodiment) preferably attaches to the cheek pad 316 via hook-and-loop fasteners, as would be understood by one of ordinary skill in the art. As such, the outside surface 350 of cheek pad 316 preferably includes either loop fasters 354, while hook fasteners reside on the strap, or vice versa. At least the inside surface 352 of check pad 316 is made of a soft-touch material, as would be understood by one of ordinary skill in the art.

A releasable engagement interface 356 on cheek pad 316 is designed to selectively engage with a clip 340. Releasable engagement interface 356 preferably includes opposing L-shaped flanges 358A, 358B and a tooth 360. L-shaped flanges 358A, 358B extend outwardly from the cheek pad 316 sufficiently to allow the lower finger 342 to slide thereunder, and serve to prevent movement of the clip 340 laterally away from the cheek pad 316. Tooth 360 preferably abuts or nearly abuts the end of lower finger 342 to prevent further longitudinal movement of the clip 340 toward the cheek pad 316.

In operation, as the lower finger 342 slides underneath the L-shaped flanges 358A, 358B, the upper finger 344 extends over top of the L-shaped flanges 358A, 358B and tooth 360. The longer length of the upper finger 344 allows it to extend past tooth 360 even as the lower finger 342 just abuts tooth 360. Further, the upper finger 344 is preferably resiliently deformable, such that it can expand or be bent away from tooth 360 to allow prong 346 to pass over top of tooth 360. Upper finger 344 then preferably returns back into its original position with prong 346 positioned past and abutting or nearly abutting with tooth 360 to prevent longitudinal movement of the clip 340 away from the cheek pad 316. In this manner, clip 340 can be engaged with releasable engagement interface 356 to secure the cheek pad 316 to the support bar 312. To remove the cheek pad 316 from clip 340, the upper finger 344 may again be expanded or bent away from tooth 360 to allow prong 346 to pass longitudinally back over top of tooth 360. This allows the upper and lower fingers 342, 344 to be withdrawn from releasable engagement interface 356.

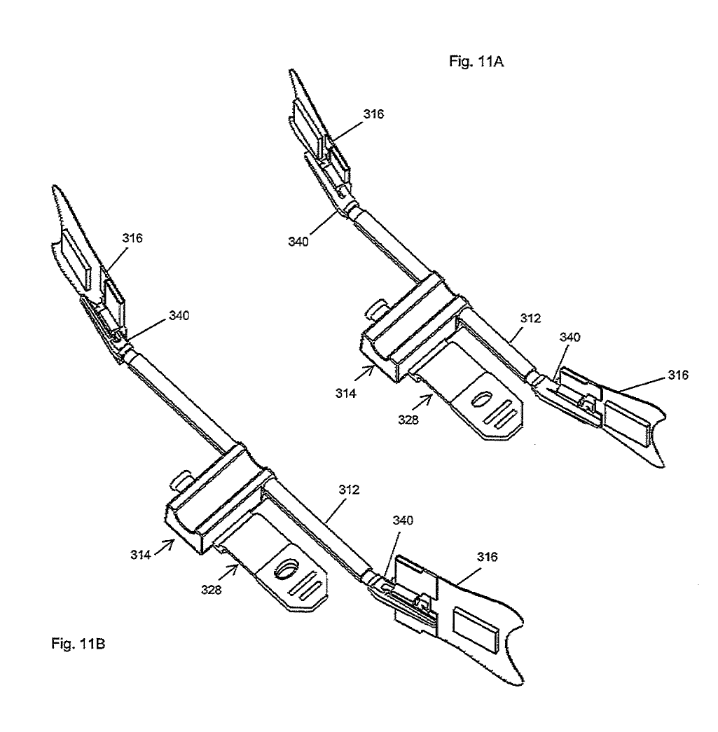

FIGS. 11A and 11B illustrate clips 340 as engaged with cheek pads 316. As can be seen, different support bars 312 may have different lengths, or the length of support bar 312 may be adjustable. Support bar 312 is preferably selectively deformable to allow it to bend around the patient's face and cheek. As such, support bar 312 preferably incudes a core composed of a material which is non-ferrous, so as not to interfere with MRIs, and will hold its position after being bent.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. An endotracheal tube stabilizer comprising:
a tube cradle;
a tube fixation element attached to said tube cradle;
a stabilization bar extending to opposing ends on either side of said tube cradle, and including an engagement element at each opposing end of the stabilization bar;
at least one cheek pad having a releasable engagement interface dimensioned to engage said engagement elements of said stabilization bar in at least one engaged position.

2. The endotracheal tube stabilizer claim 1 wherein said endotracheal tube stabilizer is a neonatal endotracheal tube stabilizer.

3. The endotracheal tube stabilizer of claim 1 wherein the stabilization bar is bendable.

4. The endotracheal tube stabilizer of claim 1 wherein a said engagement element includes upper and lower fingers, expandable one with respect to the other, for engaging with the releasable engagement interface of a said cheek pad.

5. The endotracheal tube stabilizer of claim 4 wherein said upper finger includes a prong.

6. The endotracheal tube stabilizer of claim 1 wherein the releasable engagement interface includes opposing L-shaped members and a tooth.

7. The endotracheal tube stabilizer of claim 6, wherein a said engagement element includes a lower finger for engagement between the L-shaped members for retention therein, and an upper finger having a prong for extending beyond and abutting said tooth to selectively prevent withdrawal of said lower finger from between said L-shaped members.

8. The endotracheal tube stabilizer of claim 1, wherein a cheek pad is selectively connectable to a neck strap via a hook-and-loop fastener.

9. The endotracheal tube stabilizer of claim 1 wherein said stabilization bar includes a malleable rod.

10. An endotracheal tube stabilization system comprising:
a cheek pad including opposing L-shaped members and a tooth;
a tube cradle for receiving an endotracheal tube;
a strap associated with the tube cradle for securing the endotracheal tube to the tube cradle;
a support bar extending to opposing distal ends on either side of the tube cradle;
an engagement element positioned at a distal end of the support bar, the engagement element including an upper finger and a lower finger;
wherein the L-shaped members are sized and positioned to receive and selectively retain the lower finger thereunder, and wherein the upper finger is resiliently deformable so as to allow the prong to pass overtop of the tooth as desired, and to rebound to its unbent configuration to cause the prong to engage with the tooth to prevent withdrawal of the lower finger from the L-shaped members.
